(19) United States
(12) Patent Application Publication
(10) Pub. No.: US 2011/0319769 A1
(43) Pub. Date: Dec. 29, 2011

(54) ISCHEMIC STATUS MONITORING

(75) Inventors: Sven-Erik Hedberg, Kungsangen (SE); Tomas Svensson, Stockholm (SE); Michael Broomé, Ekero (SE); Karin Järverud, Solna (SE); Stefan Hjelm, Balsta (SE)

(73) Assignee: ST. Jude Medical AB

(21) Appl. No.: 13/255,460

(22) PCT Filed: Mar. 13, 2009
(86) PCT No.: PCT/SE2009/000140

§ 371 Date: Sep. 8, 2011

(51) Int. Cl. A61N 1/365; A61B 5/026

(52) U.S. Cl. 600/481; 607/17

(57) ABSTRACT

An ischemia monitoring system has detectors for detecting the onset of an ischemic event of a tissue in subject, the end of the ischemic event and the end of a following recovery from the ischemic event, respectively. A time processor determines the duration of the ischemic event and the recovery period based on the detected onset and end times. A status processor co-processes the two determined time durations for the purpose of monitoring the ischemic status of the subject and detecting any deterioration in ischemic status for the latest ischemic event as compared to previous ischemic events that have occurred in the subject's tissue.

### BACKGROUND OF THE INVENTION

1. Field of the Invention

The present invention generally relates to ischemia monitoring, and in particular to tissue ischemic status monitoring.

2. Description of the Prior Art

Ischemia is the lack of oxygen supply to the cells. In animals, including humans, the underlying cause of ischemia is typically a cardiovascular disease, where blood vessels may be affected by arteriosclerosis. Cardiac ischemia is caused by restriction of blood flow in the coronary arteries, e.g. due to atherosclerosis. This reduced blood flow and the resulting lack of oxygen to the myocytes in the heart may lead to several effects, including hypokinesia, dyskinesia, akinesia and hibernating cells. These various effects may in turn decrease the hemodynamic performance of the heart, which ultimately can cause cardiac asynchrony, worsening heart failure and further decrease in pumping capacity.

Ischemic heart disease (IHD) is very common and approximately 7% of the total U.S. population suffers from IHD, with similar rates in other western countries.

IHD may be symptomatic, such as in angina pectoris, usually occurring suddenly and causing the patient to experience severe discomfort and pain. However, a majority of ischemic periods are silent and therefore hard to detect and classify. Most ischemic episodes, regardless of being symptomatic or silent, are reversible but still influence the risk of arrhythmias and the functional state of the heart.

U.S. Pat. No. 6,277,082 discloses detection of ischemic biological tissue by temporarily altering the temperature of the tissue and then monitoring the thermal profile of the tissue as it returns to normal temperature. Not only can ischemia be detected using such thermal profile monitoring but also the progress of recovery of the ischemic tissue can be monitored over time.

Ischemia detection is well known in the art as exemplified with the above-identified U.S. patent. However, there is still a need for a technology allowing detection and monitoring of a deterioration of ischemic status in a patient, for instance by detecting a significant deterioration of an ischemic tissue, in order to combat and possibly avoid complications and further harmful effects to the tissue.

### SUMMARY OF THE INVENTION

It is a general objective of the invention to provide ischemic status monitoring in a subject.

It is a particular objective to provide a detection of a change in ischemic status in a subject.

Briefly, in an embodiment an ischemia monitoring system according to the invention has an ischemia onset detector arranged for detecting the onset of an ischemic event in a tissue of a subject, preferably a mammalian subject and more preferably a human subject. A corresponding ischemia end detector is provided for detecting the end of the ischemic event, which also corresponds to the onset of a following recovery period. The ischemia monitoring system further has a recovery end detector for detecting the end of the recovery from the ischemic event when normal tissue function is restored. These detected onset and end times are processed by a time processor in order to determine an ischemic time interval representing the duration of the ischemic event. The time processor also determines the duration of the following recovery period in the form of a recovery time interval. The two time intervals are further co-processed for the purpose of monitoring the ischemic status of the subject and detect any change, in particular deterioration, of the ischemic burden in the tissue as compared to previous ischemic events that have been detected and analyzed for the subject's tissue.

In the embodiments, it is important to classify ischemic events and monitor the ischemic status based not solely on the duration of the ischemic event. In clear contrast, the duration of the following recovery period is also very important to analyze together with the ischemia event duration in order to achieve a correct and clinically relevant ischemic status monitoring. For instance, two ischemic events having the same duration are different from tissue function point of view if the respective following recovery periods have different durations.

The ischemic monitoring system can be implemented in an implantable medical device, such as pacemaker, defibrillator or cardioverter or indeed in a dedicated implantable ischemia monitoring device, or be partly implemented in such an implantable medical device and partly provided in a non-implantable data processing device capable of conducting communication with the implantable medical device. Alternatively, the ischemic monitoring system is solely provided in one or more non-implanted devices.

An embodiment relates to an ischemia monitoring method that detects onsets and ends of ischemic events and following recovery periods for the purpose of determining the ischemic event and recovery durations. These time durations are co-processed in order to effect the monitoring of the subject's ischemic status.

### BRIEF DESCRIPTION OF THE DRAWINGS

FIG. 1 is a schematic block diagram of an ischemia monitoring system according to an embodiment.

FIG. 2 is a schematic block diagram of an ischemia monitoring system according to another embodiment.

FIG. 3 is an overview of a subject having an implantable medical device capable of communicating with a non-implanted programmer.

FIG. 4 is a schematic block diagram of an implantable medical device according to an embodiment.

FIG. 5 is a diagram illustrating a set of ischemia severity curves.

FIGS. 6A to 6D are diagrams illustrating statistical distributions of ischemic time intervals and associated recovery time intervals.

FIGS. 7A to 7D illustrate surface ECG for healthy and ischemic heart tissue.

FIG. 8 is a flow diagram illustrating an ischemia monitoring method according to an embodiment.

FIG. 9 is a flow diagram illustrating an additional, optional step of the ischemia managing method in FIG. 8.

FIG. 10 is a flow diagram illustrating an additional, optional step of the ischemia managing method in FIG. 8.

FIG. 11 is a flow diagram illustrating an additional, optional step of the ischemia managing method in FIG. 8.

FIG. 12 is a flow diagram illustrating an embodiment of the monitoring step in the ischemia managing method in FIG. 8.

FIG. 13 is a diagram illustrating predefined ischemic time and recovery time intervals for different ischemic statuses.

### DESCRIPTION OF THE PREFERRED EMBODIMENTS

Throughout the drawings, the same reference numbers are used for similar or corresponding elements.

The present invention generally relates to ischemia and ischemia monitoring in an animal subject, preferably a mammalian subject and more preferably a human subject. Embodiments as disclosed herein are in particular suitable for detection of a significant change, such as deterioration or improvement, in the ischemic status of the subject.

Ischemic status as used herein relates to an ischemic event or attack having previously occurred in the subject's tissue. The ischemic status is further representative of the severity of the ischemic event in the tissue and the potential subsequent temporary or permanent effects the ischemic event may cause in the tissue. Thus, ischemic status can be used to denote the intensity and burden of an ischemic event and may depend on various factors, such as the blood supply and capacity status of the tissue during and following the ischemic event.

In the following, embodiments will be further described in connection with an ischemic event occurring to a subject's heart as an illustrative tissue type. However, the embodiments are not limited to heart tissue but also encompass other tissues and organs, which can suffer from ischemia. In addition to hearts and their myocytes, such potential ischemic tissue include brain tissue, in particular nerve cells; liver tissue, e.g. hepatocytes; pancreas and renal tissue, including cells in the renal medulla; intestinal tissue and in particular cells in the intestinal mucosa.

The embodiments herein allow monitoring and determining ischemic status of the subject and can be used for detecting a significant change in the ischemic status, such as a deterioration or indeed an improvement in the ischemic status. In the former case, a deterioration in ischemic status implies that a recently occurred ischemic event is regarded has having a more severe effect to the subject's tissue, such as heart, and the operation thereof as compared to a previous ischemic event. Deleterious ischemic status should be interpreted herein as an ischemic event that has a larger risk of permanent damages to the tissue and/or larger risk for further complications, such as myocardial infarctions in the case of ischemic heart tissue, as compared to less severe ischemic events. An improvement in ischemic status indicates that a recently occurred ischemic event is regarded has having less harmful effects to the subject's heart and its operation as compared to previously occurred ischemic events. Embodiments as disclosed herein can therefore trend ischemia development and such trends are followed over time as ischemic events occur to the subject's tissue.

The ischemic status monitoring and determination is achieved through the determination of two different time parameters relating to an ischemic event. These time parameters include the time interval of the ischemic event, denoted Tᵢ herein. Tᵢ, thus, reflects the time from a detected onset of an ischemic event up to a detected end of the ischemic event. The other time parameter is the recovery time of the ischemic event, denoted Tᵣ herein. Tᵣ represents the time from the detected end of the ischemic event and thereby start of the following recovery time up to the detected end of the recovery period. Ischemia recovery is sometimes denoted myocardial stunning reflecting the reversible reduction of function of heart contraction after reperfusion not accounted for by tissue damage or reduced blood flow. Thus, in the recovery period, the pumping performance of the heart is altered due to the fact that the biochemistry of the heart has to be restored following the now ended ischemic event.

In clear contrast to prior art ischemic status monitoring solutions limited to the usage of the ischemic time interval Tᵢ, the embodiments herein use not only Tᵢ but also the recovery time interval Tᵣ to obtain a relevant and accurate ischemic status monitoring and determination. Thus, the inventors have discovered that not only the duration of an ischemic event but also the duration of its following ischemia recovery should be used to achieve the accurate ischemic status determination that is needed in patient diagnosis and treatment optimization. Addition of Tᵣ in the ischemic status monitoring allows detection of ischemia intensity changes that negatively effects the operation of the heart but is not necessarily associated with prolonged ischemic time intervals. Furthermore, so-called unstable ischemic events can be detected through the combined processing of Tᵢ and Tᵣ as is further disclosed herein.

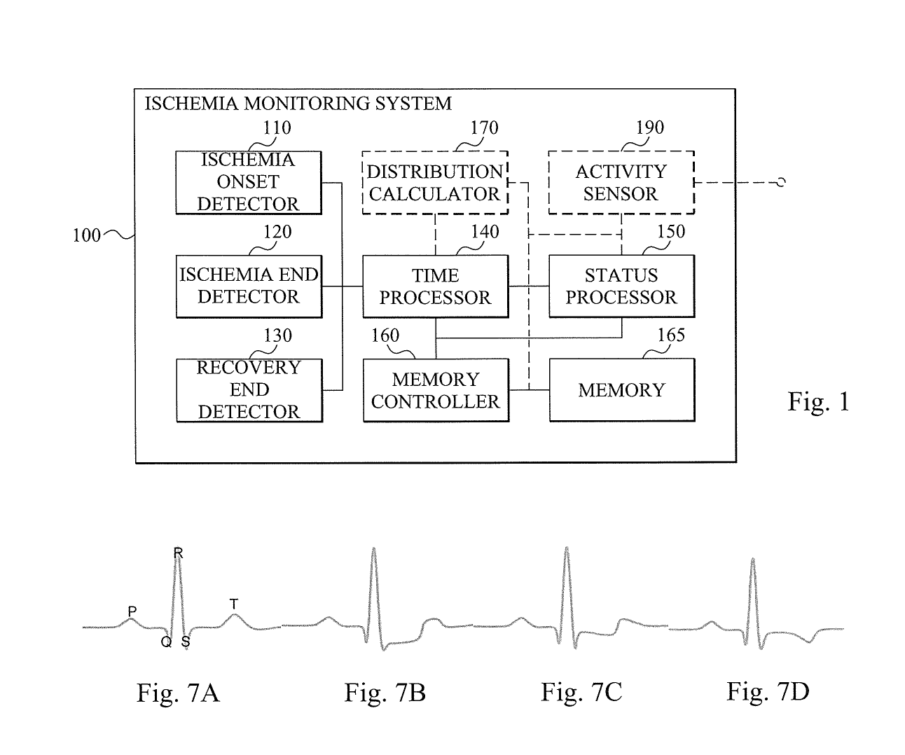

FIG. 1 is a schematic block diagram of an ischemia monitoring system 100 according to an embodiment. The monitoring system 100 includes an ischemia onset detector 110 arranged for detecting an onset of an ischemic event of a heart in a subject. The detection of the ischemia event onset triggers the start of an ischemia timer or the notification of the ischemia start time Tᵢ^{st} by a connected time processor 140. An ischemia end detector 120 of the ischemia monitoring system 100 investigates whether the detected ischemia event has ceased or whether it is still proceeding. Detection of the end of the ischemic event by the ischemia end detector 120 causes the time processor 140 to stop the ischemia timer or notify the ischemia end time Tᵢ^{e}. The time processor 140 optionally starts a recovery timer once the ischemia end detector 120 detects the end of the ischemic event. The ischemia monitoring system 100 also has a recovery end detector 130 arranged for monitoring the recovery progression of the heart following the end of the ischemic event. The recovery end detector 130 in particular detects and notifies the time processor 140 of the end of the recovery period and when correct, normal heart function has returned. Detection of the end of the recovery triggers the time processor 140 to stop the recovery timer or notify the recovery end time Tᵣ^{e}.

The time processor 140 determines an ischemic time interval of the ischemic event based on the onset of the ischemic event detected by the ischemia onset detector 110 and the end of the ischemic event detected by the ischemia end detector 120. For instance, the time processor 140 can determine the ischemic time interval Tᵢ by reading the stopped ischemia timer. Alternatively, the time processor 140 calculates the ischemic time interval as Tᵢ=Tᵢ^{e}−Tᵢ^{st}.

Correspondingly, the time processor 140 is also arranged for determining a recovery time interval based on the end of the recovery detected by the recovery end detector 130 and the end of the ischemic event detected by the ischemia end detector 120. In an embodiment, the time processor 140 determines the recovery time interval Tᵣ directly from the recovery timer, whereas in another embodiment, the time processor 140 calculates the recovery time interval as Tᵣ=Tᵣ^{e}−Tᵢ^{e}.

The two determined time parameters Tᵢ, Tᵣ are preferably forwarded to a memory controller 160 having access to a connected memory 165 implemented in the ischemia monitoring system 100 as illustrated in FIG. 1 or provided remotely but accessible to the memory controller 160. The time parameters are either directly forwarded from the time processor 140 to a connected status processor 150 or is later retrieved from the memory 165 by the memory controller 160 and provided to the status processor 150.

The status processor 150 co-processes the time parameters for the purpose of determining an ischemic status of the subject relating to the previously occurred and now recovered ischemic event. Thus, the embodiments as disclosed herein involves an ischemia monitoring system and a status processor 150 that determines the subject's ischemic status based on both the ischemic time interval and the associated recovery time interval.

The co-processing of Tᵢ, Tᵣ can be implemented according to various embodiments as is shown by the following illustrative but non-limiting examples. In general, two ischemic events or attacks having the same duration, Tᵢ, but with two different recovery time intervals Tᵣ typically reflect different ischemic status. Thus, the ischemic event with the longer recovery period is characterized to be more severe since the heart needs a comparatively longer period of time for fully or at least partly recovering from the ischemic attack and restoring normal or next to normal heart operation, including blood pumping, as compared to the ischemic event with shorter recovery period. In an embodiment, the status processor 150 may therefore calculate a parameter derivable from Tᵢ, Tᵣ. For instance, the quotient between the two time parameters can be determined

$$\frac{{T}_{i}}{{T}_{r}} or \frac{{T}_{r}}{{T}_{i}}$$

and used as status parameter, preferably together with at least one of Tᵢ and Tᵣ. Thus, the status processor 150 can calculate the quotient of the two time parameters and use it together with both or at least one of the time parameters for determining the current ischemic status of the subject and detect any change in the ischemic status.

A further example is to calculate the derivable parameter as a weighted combination of Tᵢ and Tᵣ, wᵢTᵢ+wᵣTᵣ, where wᵢ and wᵣ denotes respective weights used for the time parameters. In an implementation wᵢ=wᵣ, basically relaxing the need for any weights, whereas in preferred implementations wᵢ=1−wᵣ and 0<wᵣ<1. The actual values for these weights can be determined and set by a physician in order to reflect the importance of the ischemic period and the following recovery period to the parameter and the ischemic status determination. This allows the physician to use patient specific ischemia status classifications that more correctly reflects the underlying cardiovascular disease of the patient. Thus, for a first group of patients having a certain cardiovascular disease long recovery times with inferior cardiac pumping could be more severe as compared to other patients that can better handle longer recovery periods due to better general heart status prior the ischemic event as compared to the first group of patients. The weighted sum of the two time parameter is advantageously used in combination with one or both of Tᵢ, Tᵣ by the status processor 150 in the ischemic status determination.

The parameter derivable from Tᵢ, Tᵣ by the status processor 150 may be forwarded to the memory controller 160 for entry in the memory 165. Thus, the memory 165 could store only the “raw” time parameter Tᵢ, Tᵣ, these time parameters and the parameter derivable therefrom or indeed only the derived parameter.

The status processor 150 preferably detects a change in ischemic status of the subject based on Tᵢ, Tᵣ. In such a case, the status processor 150 has access to one or more reference parameters that is/are compared to the time parameters Tᵢ, Tᵣ or the one or more parameters derivable from Tᵢ, Tᵣ. The ischemia onset detector 110 then preferably detects the onset of not only one but multiple, i.e. at least two, different ischemic events occurring in the heart of the subject. The ischemia end detector 120 and the recovery end detector 130 correspondingly detect the end of the ischemic events and the end of the following recovery periods, respectively. This means that the time processor 140 calculates N multiple pairs of Tᵢ, Tᵣ for the N different ischemic events. The respective time parameters are preferably entered in the memory 165 by the memory controller 160 as they are determined by the time processor 140.

The status processor 150 can then calculate respective derivable parameters, such as quotients or weighted sums, from the N pairs of time parameters Pₖ. The trend of these N parameters Pₖ is preferably followed and used by the status processor 150 for detecting a significant change in ischemic status. For instance, the status processor 150 calculates a reference parameter \hat{P} based on N−1 previously calculated parameter Pₖ, such as an average of the N−1 parameters and more preferably a weighted average,

$$\hat{P} = \frac{1}{N - 1} {\sum }_{k = 1}^{N - 1} {w}_{k} {P}_{k}$$

with

$${\sum }_{k = 1}^{N - 1} {w}_{k} = 1$$

and preferably w_{N-1}>w_{N-2}> . . . >w₁. Such weighted average put more weights to more recently determined time parameters Pₖ as compared such time parameters determined for older ischemic events.

The status processor 150 compares the newly determined parameter P_{N} with the calculated average parameter \hat{P} for the purpose of detecting a significant change in the ischemic status of the subject. Thus, if P_{N} differs with more than a threshold valued from \hat{P}, the status processor 150 concludes that a ischemic status change has been detected and that the intensity or burden of the recently occurred ischemic event significantly differs from the preceding N−1 ischemic events. In such a case, the status processor 150 can trigger further actions as is described in more detail herein.

If the status processor 150 concludes that there is no significant change in the ischemic status as determined based on the comparison, the parameter P_{N} is preferably entered in the memory 165 by the memory controller. The status processor 150 optionally also updates the average parameter \hat{P} based on P_{N}, such as

$$\hat{P} = \frac{1}{N - 1} {\sum }_{k = 2}^{N} {w}_{k - 1} {P}_{k} or \hat{P} = \frac{1}{N} {\sum }_{k = 1}^{N} {w}_{k} {P}_{k} .$$

In an alternative approach, an optional distribution calculator 170 is arranged in the ischemia monitoring system 100 for calculating a statistical distribution of the N pairs of Tᵢ, Tᵣ. The distribution calculator 170 retrieves the N−1 pair(s) of Tᵢ, Tᵣ from the memory 165 and gets the most recent Tᵢ, Tᵣ pair from the status processor 150 or indeed from the memory 165. The status processor 150 performs the ischemic status determination based on the comparison of the statistical distribution from the distribution calculator from a reference statistical distribution. This reference statistical distribution is preferably determined by the distribution calculator 170 based on previous pairs of Tᵢ, Tᵣ not including the most recent Tᵢ, Tᵣ pair.

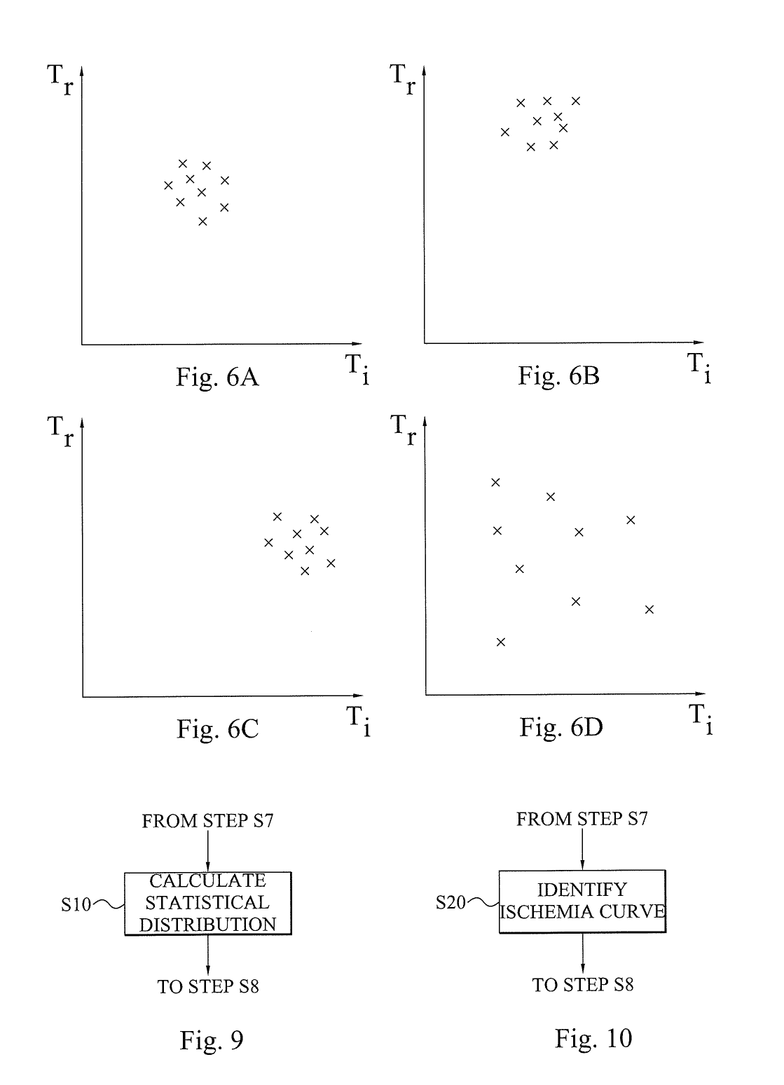

FIGS. 6A to 6D illustrate diagrams plotting pairs of Tᵢ, Tᵣ to form a distribution of points in the Tᵣ versus Tᵢ or Tᵢ versus Tᵣ diagrams. The distribution of points can be regarded as a fingerprint of the ischemic status or ischemic burden of the subject's heart. FIG. 6A represents a baseline case, where a subject has a stable ischemic status resulting in no significant change in ischemic time intervals and recovery time intervals. FIG. 6B illustrates a subject having ischemic events with comparatively longer recovery time intervals as compared to FIG. 6A, whereas FIG. 6C illustrates a corresponding distribution of Tᵢ, Tᵣ points for a subject with longer ischemic time intervals. If a subject has an initial distribution of Tᵢ, Tᵣ points as illustrated in FIG. 6A but subsequent cardiac ischemic events result in a significant change in recovery time interval, as in FIG. 6B, and/or a significant change in ischemic time interval, as in FIG. 6C, the ischemic status of the subject is changed. In both these cases, a deterioration or worsening of the ischemic burden has occurred as compared to the baseline condition of FIG. 6A.

FIG. 6D illustrates a subject suffering from unstable ischemic attacks having vastly varying ischemic time intervals and recovery time intervals. Such unstable ischemic status is believed to be particularly severe and can be a sign of an unstable coronary vascular disease. The ischemic status monitoring and determination of the embodiments can therefore be used for identifying subjects having unstable ischemic events and underlying diseases, thereby being of particular large risk of irreversible damage to the myocardium and myocardial infarction.

In a particular embodiment, the distribution calculator 170 calculates at least one statistical parameter representative of the distribution of Tᵢ, Tᵣ pairs. The at least one statistical parameter can advantageously be in the form of an average of the respective Tᵢ, Tᵣ values and preferably the standard deviations. In such a case, such statistical parameters are first calculated by the distribution calculator 170 based on the previously determined Tᵢ, Tᵣ pairs stored in the memory. The same statistical parameters are further calculated once more but also including the Tᵢ, Tᵣ pair determined by the time processor 140. If there is a significant change of the mean value and/or the standard deviation, the status processor 150 determines that that the ischemic status of the subject has changed. This embodiment is however not limited to usage of mean value and standard deviation but can instead be used in connection with any statistical parameter or set of parameters representative of a distribution of multiple Tᵢ, Tᵣ pairs. Example of such other parameters is to calculate respective quotients of Tᵢ, Tᵣ in order to form a distribution.

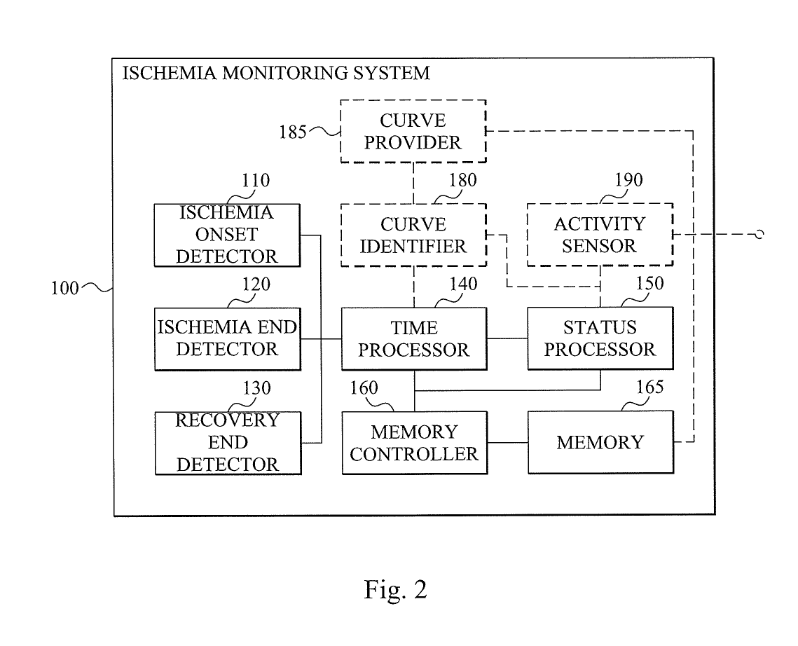

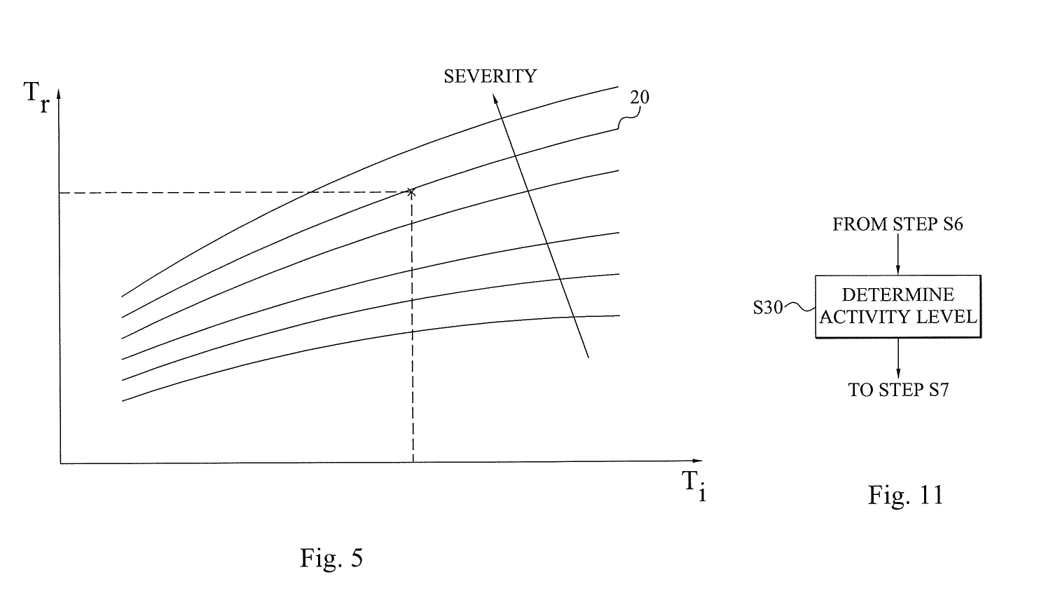

FIG. 2 is a schematic block diagram of another embodiment of the ischemia monitoring system 100 having functionality for determining the ischemic status of a subject by means of another technique than distribution calculations. This embodiment has a set of so-called severity curves stored in the memory 165. These severity curves lists or plots Tᵣ for different Tᵢ values or vice versa. Furthermore, each such severity curve has particular relationships between the recovery time interval and the ischemia time intervals so that the multiple severity curves have different recovery time intervals for each ischemic time interval. FIG. 5 illustrates a diagram with multiple such severity curves. Each such severity curve 20 has an associated ischemia severity classification representative of the ischemic status for different pairs of Tᵢ, Tᵣ. As is seen in FIG. 5, the ischemia severity classification typically goes from less sever ischemic events towards more severe ischemic events when traveling between the curves 20 along the illustrated arrow. Thus, the more severe ischemia event, the longer the recovery time interval for a given ischemia time interval.

A curve provider 185 is arranged in the ischemia monitoring system 100 for providing the multiple ischemia severity curves. This curve provision is preferably implemented by fetching the respective Tᵢ, Tᵣ values for the curves from the memory 165. These curves can have been pre-defined, e.g. by a physician and entered or downloaded in the memory 165 using the memory controller 160. Also patient-specific ischemia severity curves can be used by the embodiments. In such a case, each time the time processor 140 determines a pair of Tᵢ, Tᵣ following detection of an ischemic event, the ischemic event is further assigned an ischemic severity classification, for instance by the physician. Once the patient has had multiple ischemic attacks, several Tᵢ, Tᵣ pairs may be determined for different ischemic severity classifications, thereby forming a data set that can be used to define the multiple ischemia severity curves.

It should be noted that in some applications no dedicated curves need to be drawn or analyzed. In clear contrast, a set of Tᵢ, Tᵣ pairs is together defined as constituting or forming an ischemia severity group or curve having an assigned ischemia severity classification. Multiple such sets of Tᵢ, Tᵣ are then stored in the memory 165.

A curve or group identifier 180 is also arranged in the ischemia monitoring system 100 in this embodiment for identifying an ischemia severity curve or group provided by the curve or group provider 185. This identification is furthermore performed based on the Tᵢ, Tᵣ pair determined by the time processor 140 for the detected and now lapsed ischemic event. The status processor 150 determines the ischemic status of the subject based on the ischemia severity classification assigned to the ischemia severity curve or group identified by the curve identifier 180.

FIG. 5 graphically illustrates this curve identification by plotting, together with the multiple ischemia severity curves, a determined Tᵢ, Tᵣ pair. The curve 20 that is closest to the coordinate defined by the Tᵢ, Tᵣ pair is identified and the ischemia severity classification assigned to this curve 20 is used by the status processor 150 for the purpose of determining the ischemia status of the subject. The curve identification could be implemented by identifying the curve 20 that minimizes the Euclidean distance between the Tᵢ, Tᵣ coordinate and a point on the curve 20.

An alternative embodiment is illustrated in the diagram of FIG. 13. In this embodiment, the ischemia monitoring system 100 has access to predefined threshold values Tᵢ¹⁻³, Tᵣ^{1,2} associated with different ischemic severity classifications. These thresholds have been illustrated in FIG. 13 and defines, in the Tᵢ, Tᵣ plane, different sub-regions having different ischemic severity classification. As is illustrated in the figure, previously recorded Tᵢ, Tᵣ have mainly been present in the sub-region in which Tᵢ¹≦Tᵢ<Tᵢ² and Tᵣ¹≦Tᵣ<Tᵣ². The three ischemic events resulting in the three Tᵢ, Tᵣ value pairs present in this sub-region therefore has a same ischemia severity classification. However, a subsequent ischemic event results in significantly prolonged recovery period and ischemic period and therefore falls within another sub-region, Tᵢ²≦Tᵢ<Tᵢ³ and Tᵣ²¹≦Tᵣ. This latter ischemic event is consequently assigned another ischemia severity classification by the status processor 150 in the ischemia monitoring system.

The threshold values used to define the different sub-regions are stored in the memory 165 and are preferably defined by a physician and entered by him/her in the memory 165. Also patient-specific thresholds can be defined if the patient has suffered from multiple previous ischemic events that have been classified as disclosed herein. In such a case, these prior classifications and the determined Tᵢ, Tᵣ values can be used to generate the necessary information, i.e. thresholds, to define the sub-regions with different ischemia severity classification.

The respective numbers of thresholds illustrated in FIG. 13 should merely be seen as an illustrative but non-limiting example.

The embodiments of the ischemia monitoring system 100 illustrated in FIGS. 1 and 2 may optionally further comprise an activity level sensor 190. There are several different activity sensors 190 known in the art that can be used according to the embodiments. For instance, accelerometers, motion transducers, including piezo-based transducers, heart rate sensors, respiratory rate sensors, respiratory depth sensor etc. can be used and are all well-known in the art. The activity sensor 190 is preferably arranged for estimating an activity level of the subject at least partly during the ischemic event and/or during the following recovery period. For instance, the activity sensor 190 can be arranged for determining the activity level of the subject in connection with at least one of the ischemia onset detector 110 detecting the onset of the ischemic event, the ischemia end detector 120 detecting the end of the ischemic event and the recovery end detector 130 detecting the end of the recovery from the ischemic event.

The activity level data determined by the activity sensor 190 is either entered in the memory 165 or is directly forwarded to the status processor 150. In either case, the status processor 150 uses this activity data together with the ischemic time interval and the recovery time interval in order to determine the ischemic status of the subject. Usage of activity data in connection with the time and recovery time intervals may lead to more accurate status determination and, in particular, to detecting significant deteriorations in the ischemic burden of the subject. The reason for this is that at a high activity level, there is a large oxygen demand by the myocardium. This large oxygen demand might not be fully supplied due to a cardiovascular disease, such as atherosclerosis, thereby causing the initiation of an ischemic event for at least a part of the heart. If the subject, however, would not have been exercised or otherwise have had such high activity, the ischemic event might not have occurred or would have been milder. Furthermore, an ischemic event and following recovery period resulting in a particular pair of Tᵢ, Tᵣ is generally regarded as being more severe if the activity level of the subject is normal or low as determined by the activity sensor 190 when compared to an ischemic event leading to the same pair of Tᵢ, Tᵣ but during a period of high patient activity. Thus, the status processor 150 preferably utilizes the activity level data together with Tᵢ, Tᵣ in the ischemic status determination or at least preferably notifies and stores the average patient activity during the ischemic and/or recovery periods and in particular the ischemic periods together with the Tᵢ, Tᵣ data.

The units 110 to 190 of the ischemia monitoring system 100 may be implemented in hardware, software or a combination of hardware and software. The ischemia monitoring system 100 may be implemented in an implantable medical device or in a non-implantable data processing device, such as a computer or computer system, as is further described herein. A distributed implementation is also possible with some of the units implemented in a first device, such as an implantable medical device, and with the other units provided in a second device, such as programmer, capable of conducting wired or wireless communication with the first device. For instance, the ischemia onset detector 110, ischemia end detector 120, recovery end detector 130 and optionally the time processor 140 can be arranged in the first device with the remaining units, possibly excluding the optional activity sensor 190, arranged in the second, non-implanted device.

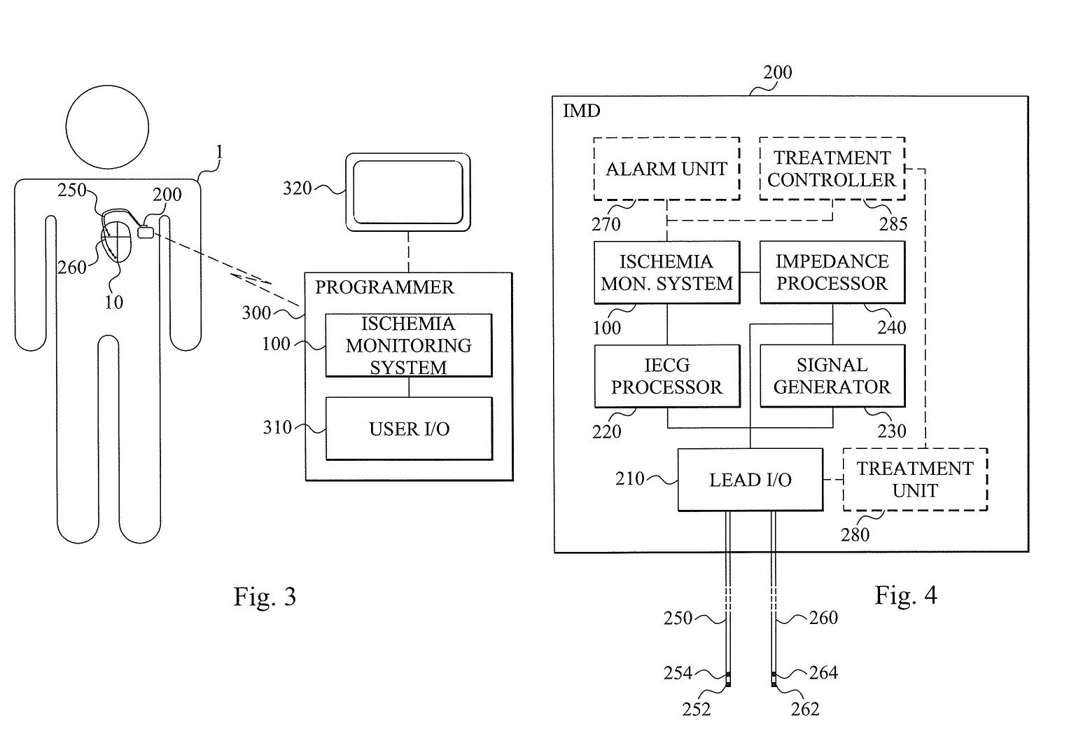

FIG. 3 is a schematic overview of a human patient 1 having an implantable medical device (IMD) 200. In the figure, the IMD 200 is illustrated as a device that monitors and/or provides therapy to the heart 10 of the patient 1, such as a pacemaker, cardiac defibrillator or cardioverter. The IMD 200 is, in operation, connected to one or more, two in the figure, cardiac leads 250, 260 inserted into different heart chambers, the right atrium and the right ventricle in the figure, or being epicardially positioned relative the heart 10. The IMD 200 must not necessarily be connected to two cardiac leads 250, 260 but could alternatively be connected to a single lead 260 carrying at least one electrode or more than two cardiac leads 250, 260.

FIG. 3 also illustrates an external programmer or clinician's workstation 300 that can communicate with the IMD 200. As is well known in the art, such a programmer 300 can be employed for transmitting IMD programming commands, using an included transmitter (not illustrated), causing a reprogramming of different operation parameters and modes of the IMD 200. Furthermore, the IMD 200 can upload diagnostic data descriptive of different medical parameters or device operation parameters collected by the IMD 200 to a receiver (not illustrated) of the programmer 300. Such uploaded data may optionally be further processed in the programmer 300 before display to a clinician on a connected display screen 320.

In a particular embodiment, the ischemia monitoring system 100 such as disclosed in FIG. 1 or 2 can be implemented or at least partly implemented in the programmer 300 or indeed in another non-implantable data processing device. In such a case, the ischemia monitoring system 100 can receive data input allowing the including units to identify ischemia onset, end and recovery end. This input can be affected by a general input and output (I/O) 310 of the data processing device 300. This means that the I/O 310 could be connected to further equipment that can be used for the purpose of collecting data that can be processed by the ischemia onset detector, ischemia end detector and/or the recovery end detector. Alternatively, the I/O 310 is a so-called user I/O 310 that can be utilized by a user, such a physician, for manually inputting data that are processed by the ischemia monitoring system 100 for ultimately determining the ischemia time interval and the recovery time interval.

Herein follows more detailed examples of how the ischemia and recovery time intervals can be determined using non-implantable devices. These devices can therefore constitute a part of the data processing device, be connected thereto for automatic or user-triggered transfer of data or be separate from the data processing device, in which case a user needs to manually input to using the user I/O 310.

A preferred technique for determining the ischemia time interval is to use a surface electrogram (ECG) and in particular through ST segment analysis of such surface ECG. The technique involves recording a surface ECG during at least one cardiac cycle, preferably of multiple consecutive cardiac cycles. In the latter case, one or more of these multiple consecutive cardiac cycles can be investigated further as disclosed herein. Alternatively, an average cardiac cycle is first generated based on the multiple consecutive cardiac cycles in order to reduce the impact of spontaneous deviations, noise and other interfering sources.

The so-called R wave of a QRS complex in a (average) heart cycle is identified, preferably as corresponding to the ECG value having the largest absolute value. A time window following the localized R wave in the cardiac cycle is defined. This time window should cover the ST segment and preferably the T wave of the heart cycle. For a human with a normal heart rate, this time window is typically 50-300 ms after the R wave. However, depending on the age and activity level of the subject, among others, the size and location of the time window can be different. The T wave can be identified as corresponding to a local max (in the case of normal ECG) following the end of the QRS complex.

FIGS. 7A to 7D schematically illustrate surface ECG of a portion of a heart cycle, clearly depicting the QRS complex and the P and T waves. FIG. 7A is the ECG from a healthy heart. FIGS. 7B and 7C illustrate two versions of ST segment depletions that have occurred in an ischemic heart. In FIG. 7D, the ischemic heart presents a T wave inversion on the recorded surface ECG.

The ECG level in the measurement window, i.e. the ST segment and the T wave, is measured. This ECG level can be any parameter representative of the ECG level in the measurement window. A non-limiting example is the averaged value in the measurement window.

This parameter is then compared with a reference parameter, which preferably corresponds to the ECG level in the measurement window during one or more heart cycle with normal, non-ischemic condition of the patient. The reference parameter is therefore advantageously determined in connection with a patient visit at a physician and then stored in the ischemia monitoring system for later use.

The calculated parameter is compared to the reference parameter and if there is a significant difference there between, the ischemia onset detector detects the onset of an ischemic event. The ischemia end detector correspondingly detects the end of the ischemic event once there is no longer any significant difference between the calculated parameter and the reference parameter. In order to reduce the risk of misinterpreting naturally varying parameter differences for ischemia-triggered parameter differences, the ischemia onset detector could be configured for detecting the ischemia event onset only if the respective calculated parameters for multiple, preferably consecutive, heart cycles differ significantly from the reference parameter. Correspondingly, the ischemia end detector can be configured for detecting the end of the ischemic event only if the respective calculated parameters for multiple, preferably consecutive, heart cycles no longer differ significantly from the reference parameter.

In an alternative embodiment, no parameter representative of the ECG level in the measurement window is determined. In clear contrast, the complete ECG waveform of the ECG signal in the measurement window is extracted. The extracted ECG waveform is then compared to a reference ECG waveform recorded from the subject during one or multiple heart cycles during normal, non-ischemic condition. The ECG waveform and the reference ECG waveform are compared using any type of curve or pattern comparing technique for the purpose of detecting any significant difference. For instance, the absolute difference or the squared difference between respective ECG samples in the ECG waveform and the reference ECG waveform can be summed and compared to a pre-defined threshold. If the summed absolute or squared differences exceed the threshold, a significant change is detected corresponding to a detected onset of an ischemic event. Correspondingly, once the absolute or squared differences no longer exceed the threshold, the end of the ischemic event is detected.

The recovery time interval is advantageously determined from sensor data capturing the mechanical behavior of the heart. A typical example of suitable sensor is a pulse oximeter registering the blood pressure or photoplethysmograph sensor measuring volume changes and can be used for monitoring blood pressure. Other sensing techniques that can monitor the blood pressure could alternatively be used.

In a typical setting, a heart cycle is identified from the plethysmogram, such as the portion of the plethysmogram between two consecutive R waves. This identified waveform is compared to a reference waveform determined for the patient during normal, non-ischemic condition. The photoplethysmograph sensor is preferably automatically or manually activated once the end of the ischemic period has been detected as described above. In such a case, the mechanical pumping of the heart is monitored by comparisons of the plethysmogram data with the reference waveforms until there no longer is any significant difference between the plethysmogram waveform and the reference waveform. This point in time corresponds to the end of the recovery period when the mechanical behavior of the heart has been restored.

The data processing device embodying the ischemia monitoring system can therefore advantageously be connected to a surface ECG recording unit and a plethysmograph sensor in order to provide input data to the ischemia monitoring system for the purpose of identifying the ischemia and recovery onset and end times. Alternatively, the recorded ECG data and plethysmogram data can be input to the data processing device by a user to then be analyzed by the ischemia monitoring system.

FIG. 4 is a schematic block diagram of an IMD 200 according to an embodiment. The IMD 200 has a lead or electrode connecting arrangement 210 represented by a lead input/output (I/O) 210 in the figure.

This lead I/O 210 is, in operation, connectable to multiple electrodes 252, 254, 262, 264 of which at least one is designed for being implanted in or at least in connection with the heart. As a consequence, at least one of the multiple electrodes 252, 254, 262, 264 is arranged on a cardiac lead 250, 260 connectable to the lead I/O 210. This further implies that at least one but not all of the multiple electrodes 252, 254, 262, 264 may not necessarily be lead-arranged or be implanted in the immediate vicinity of the heart. An example of such an electrode, is an electrode constituting the whole or a portion of the case or can of the IMD 200.

As is well known in the art, an implantable lead or catheter 250, 260 has a proximal end connectable to the IMD 200 through the lead I/O 210. This IMD-connecting end presents one or more electric terminals that are in electric connection with the electrodes 252, 254, 262, 264 present on the opposite distal lead end, where the electric connection is achieved by electric conductors running along the length of the lead body. The distal lead end with its electrodes 252, 254, 262, 264 is then provided in connection with the heart tissue. For this purpose, the lead 250, 260 can include a tissue anchoring element, such as a helical fixation element, though other fixation elements, such as passive fixation elements, including fines, tines, etc., are also common. The fixation element can indeed constitute one of the electrodes of the lead 252, 262, while remaining electrodes can be ring electrodes 254, 264 often denoted indifferent electrodes in the art, defibrillation electrode, or the like.

The IMD 200 is connected to at least one implantable cardiac lead 250, 260. The cardiac lead 250, 260 can be an intracardiac lead positioned in any of the chambers of the heart, such as right and/or left atrium and/or ventricle. Alternatively, the lead 250, 260 could be epicardially positioned relative the heart, such as in the coronary vein. In the case of multiple connectable leads 250, 260 the IMD 200 can be connected to multiple intracardiac or endocardial leads, multiple epicardial leads or a combination of intracardial and epicardial leads. Generally, in a single-chamber bradycardia device 200 a single cardiac lead 250 is implanted in the right ventricle of the heart. Correspondingly, in a dual-chamber cardiac resynchronization defibrillator, a respective cardiac lead is typically implemented in the right atrium, the right ventricle and epicardially relative the left ventricle.

In an embodiment, the IMD 200 includes the ischemia monitoring system 100 or at least a portion thereof as previously mentioned.

In an embodiment, the IMD 200 includes an intracardiac ECG (IECG) processor 220 arranged connected to the lead I/O 210 and arranged for generating IECG data based on electric signals collected from the heart by the electrodes 252, 254, 262, 264. The IECG data can then be analyzed by the ischemia onset detector and ischemia end detector as described above in the case of a surface ECG for the purpose of detecting the start and end of an ischemic event based on ST segment deviations.

In a particular embodiment, if the IMD 200 is connectable to one or more leads 250, 260 together having multiple electrodes 252, 254, 262, 264 implanted in or in connection with the heart, multiple separate IECGs can be recorded by the IECG processor 220 for different pairs of the electrodes 252, 254, 262, 264 and/or for different combinations of a case electrode and the cardiac-implanted electrodes 252, 254, 262, 264. These multiple IECGs are then co-processed by the IECG processor 220 for the purpose of calculating a pseudo-global IECG for the heart. This co-processing can be conducted by calculating an average of the multiple unipolar IECGs and/or the bipolar IECGs. The pseudo-global IECG provides a more global electrical fingerprint of the heart as compared to individual IECG that more reflects the local electric activity in a portion of the heart. It is expected that global electrical activity is more valuable for the purpose of ischemia detection and the determination of the ischemic time interval.

The detection of the onset and end of the ischemic time interval is then basically performed in the same way as for the surface ECG with the exception that the IECG or pseudo-global IECG is processed by the ischemia onset and end detectors instead of the surface ECG.

Recording of multiple IECGS and the generation of a pseudo-global IECG is not mandatory for the purpose of detecting the onset and end of the ischemic time interval by an IMD 200. In particular local ischemia monitoring in selected parts of the heart can benefit from using one or more individual IECGs representative of the local electrical activity of the heart over time in the selected parts.

If the IMD 200 is connected to a single lead 250, the number of cardiac electrodes can be more limited and therefore recorded IECGs could be more local as compared to an IMD 200 having two or more connected leads 250, 260. In such a case, generated IECGs from the IECG processor 220 can be further processed for the purpose of synthesizing or emulating a surface ECG from the IECGs. Techniques for such surface ECG emulation from IECGs are disclosed in the U.S. Pat. No. 6,813,514 the teaching of which regarding surface ECG emulation is hereby incorporated by reference. Briefly, the technique is based on inputting electrical cardiac signals sensed using a combination of pairs of electrodes implanted within the patient and then emulating each of a plurality of separate signals associated with a multiple-lead surface ECG of a patient based on the input electrical cardiac signals. By emulating each of the individual signals of the surface ECG rather than merely generating a combined surface ECG, the separate signals can be individually processing using a wide variety of techniques, such as filtering the individual signals separately.

The emulated surface ECG can then be used by the ischemia monitoring system 100 in similarity to the above disclosed measured surface ECG for the purpose of detecting onset and end of an ischemic event based on ST-deviations in the surface ECG.

In a preferred embodiment, the IMD 200 has a signal generator 230 electrically connected to the lead I/O 210 and connectable electrodes 252, 254, 262, 264. The generator 230 generates an electric signal. The electric signal is an alternating current (AC) signal having particular frequency or frequencies. The electric signal is applicable over at least a portion of a heart in a subject by two electrodes 252, 254 of the multiple connectable electrodes 252, 254, 262, 264.

In operation, the signal generator 230 generates the electric signal having a defined time-dependent voltage/current profile and forwards the signal to the lead I/O 210. The lead I/O 210 directs the electric signal to the two relevant signal applying electrodes 252, 254 to apply the signal over the relevant portion of the heart. Two electrodes 252, 254 of the multiple connected electrodes 252, 254, 262, 264 collect a resulting electric signal, i.e. resulting AC signal, originating from at least the portion of the heart. This resulting signal is due to the applied electric signal from the signal generator 230. In the case multiple electric signals where generated by the signal generator 230 and applied over different portions of the heart, a respective resulting electric signal is preferably collected by respective electrode pairs for each of the applied electric signals.

An impedance processor 240 is electrically connected to the signal generator 230 and the lead I/O 210. The impedance processor 240 processes the electric signal generated by the signal generator 230 and the resulting electric signal collected by the two electrodes 252, 254 connected to the lead I/O 210. In more detail, the processor 240 calculates cardiogenic impedance data or signal based on the generated electric signal, such as based on the current of the electric signal, and the resulting electric signals, e.g. based on the measured voltage of the resulting electric signal. This impedance data is reflective of the mechanical function of at least a portion of the heart and more preferably representative of the mechanical pumping action of the heart.

This cardiogenic impedance data is processed by the ischemia monitoring system for the purpose of detecting the end of the recovery period. This detection is possibly as any mechanical dysfunction of the heart caused by the ischemic event and present during the recovery period can efficiently be picked up by the cardiogenic impedance. Thus, during the recovery period, the cardiogenic impedance reflective of the mechanical heart function is significantly different from the cardiogenic impedance during normal, non-ischemic/recovery periods.

In a typical implementation, a complete heart cycles is identified in the impedance data, such as between two consecutive R waves. The waveform or set of impedance samples of this portion of the impedance signal is analyzed with a corresponding waveform or set of impedance samples previously recorded during a period of normal non-ischemic and non-recovery operation. A waveform or data set comparison is then conducted as previously described for the purpose of detecting a significant difference between the waveform/set and a reference waveform/set, which difference is present during the recovery period. Once the heart function, i.e. pumping action, as been restored, corresponding to the end of the recovery period, the recovery end detector determines that there is no longer any significant difference between a waveform/set and the reference waveform/set or there is no such significant difference for multiple consecutive waveforms/sets and the reference waveform/set.

In a preferred embodiment, the waveform/set of impedance samples not necessarily corresponds to a compete heart cycle. In clear contrast, the waveform/set of impedance samples preferably corresponds to the diastolic phase, or a portion thereof, of the heart cycle. It has been concluded that this part of the heart cycles is particularly sensitive for registering changes in the cardiogenic impedance signals originating from mechanical dysfunction of the heart following an ischemic event. Therefore, the diastolic part of the impedance waveform or data set is preferably compared to the reference waveform or data set.

In the case systolic variables are easier to measure the waveform/set of impedance samples could instead correspond to the systolic phase, or a portion thereof, of the heart cycle.

The IMD 200 advantageously comprises or is connectable to a posture sensor (not illustrated) for determining a current posture of the subject. Such posture sensors adapted for implementation in an IMD 200 are well-known in the art. There is actually combined position and activity sensor available. Such a combined activity and posture sensor is then preferably used in order to obtain not only activity data but also posture data but without the drawback of having extra dedicated sensor equipment in the IMD 200. The subject posture determined by the (activity and) posture sensor is preferably collected in connection with determining the recovery time interval, in particular if an impedance-based technique is used for the recovery time interval determination. The cardiogenic impedance is generally partly affected by the current body posture, resulting in different cardiogenic impedance waveforms for a standing body posture as compared to a subject lying down, even though the same impedance vector has been used. The impedance-based recovery time interval determination is therefore preferably coordinated and associated with posture data from the posture sensor. In such a case, a set of multiple reference parameters or reference recovery time parameters can be available for different body postures, such as one for a standing body and one for a reclining body. The posture data is then used in order to select the particular reference parameter(s) to use when comparing the determined ischemic and recovery time intervals with the reference parameter(s) for the purpose of determining the ischemic status and detecting any significant change thereof. The inclusion of body posture data in the determination of the ischemic status further increases the specificity and accuracy in the determination and thereby provides an improved ischemic status determination and monitoring.

The IMD 200 may optionally include an alarm unit 270 capable of sounding an alarm signal or providing a tactile alarm signal. This alarm unit 270 is responsive to the ischemia monitoring system 100 and more particularly to the status processor detecting a deterioration in the ischemic status of the subject as previous described. This alarm will alert the subject of the lapsed ischemic event and urge him/her to contact his/her physician and/or take suitable anti-ischemic medicament, such as nitrates, beta-blockers, calcium channel blockers, aspirin, etc. In addition, or alternatively, the alarm unit 270 or some other unit in the IMD 200 may generate an alarm signal that is automatically uploaded to an external communication unit, such as home monitor, mobile telephone, personal digital assistant, computer, or other unit capable of wirelessly receiving data from the IMD 200. This external unit may optionally display a warning message on a display screen informing the IMD patient of the detected deterioration in ischemic status. Furthermore, or instead, the external unit forwards the alarm signal to the physician using a computer network, telephone network or a radio-based wireless communication network.

The IMD 200 may optionally include a so-called treatment unit 280 arranged connected to the lead I/O 210 for generating electric treatment signals or pulses to a portion of the heart using electrodes 252, 254, 262, 264 of the at least one connectable lead 250, 260. The treatment unit 280 generates the electric treatment signal according to a treatment scheme, defining the duration of the electric treatment pulses, the timing thereof and other characteristics of the electric treatment signal.

The treatment unit 280 is preferably connected to and controlled by a treatment controller 285. This treatment controller 285 can, for instance, trigger the treatment unit 280 to start generating a particular electric treatment signal, switch to another treatment scheme to use for the purpose of generation and application of the electric treatment signals and/or adjust particular settings, such a pulse duration, timing, pulse magnitude, etc.

In a particular embodiment, the treatment controller 285 is responsive to the ischemia monitoring system 100 and implemented for controlling the operation of the treatment unit 280 based on the detection of a change in the ischemic status of the subject as determined by the status processor of the ischemia monitoring system 100. The treatment controller 285 is advantageously arranged for selecting an anti-ischemia treatment scheme or adjusting a treatment scheme currently used by the treatment unit 280 based on the detection of a deterioration of the ischemic status of the subject. A preferred such anti-ischemia treatment scheme adjustment is to reduce the maximum tracking rate of the treatment unit 280 to thereby reduce the pacing rate. A reduced pacing rate (ventricular contraction rate) will improve the coronary perfusion, which in turn improves the ischemic status.

Other anti-ischemia treatment actions could alternatively, or in addition, be triggered by the treatment controller 285, such as the release of anti-thrombotic agent if the IMD 200 includes equipment for allowing a controlled release of such agents.

The units 100, 210-240, 270-285 of the IMD 200 may be implemented in hardware, software or a combination of hardware and software. In FIG. 4 and the discussion of embodiments above, reference has only been to the IMD unit directly involved in the present invention. It is therefore expected according to the invention that the IMD 200 will typically include other units and functionalities required for efficient and correct operation of the IMD 200.

The present embodiments are not limited to the above presented techniques for detecting the onset and end of the ischemia event and recovery period. In clear contrast, other techniques known in the art for detecting ischemia onset and end and recovery onset and end can of course be used in connection with the present embodiments. For instance, biochemical markers, such as Troponin I/T, NT-proBNP and local potassium leakage can be used for ischemia time interval determination. Correspondingly, other techniques for monitoring the mechanical function of the heat, such as recovery of left ventricular diastolic pressure (LVDP), left atrial pressure (LAP), pulmonary arterial oxygen saturation can be used for the purpose of recovery time interval determination.

In the brain, the ischemic time interval can be determined from blood pressure measurements with a blood pressure sensor. Alternatively, blood and tissue oxygen changes detected by oxygen sensors can be used. The following recovery time interval is advantageously determined from electroencephalography (EEG) measurements. Detection of the end of the recovery period is then identified based on waveform analysis between recorded EEG waveform and a reference waveform in similarity to (I)ECG analysis.

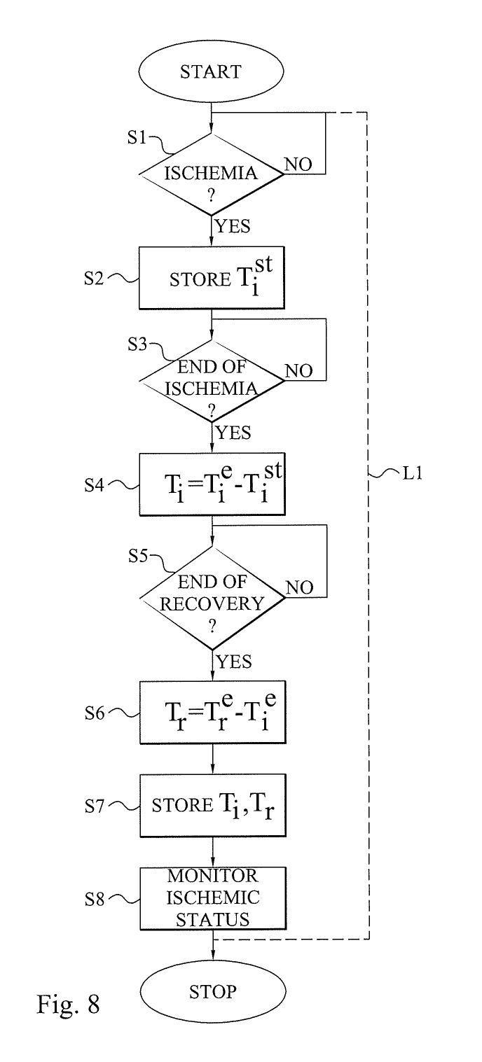

FIG. 8 is a flow diagram illustrating an ischemia monitoring method according to an embodiment. The method generally starts in step S1, which investigates whether an ischemic event is detected. If such an even is detected the method continues to step S2, where the start time Tᵢ^{st} of the ischemic event is stored or an ischemia trigger is started. A next step S3, typically periodically, investigates whether the detected ischemic event has ended, thereby continuing the method to step S4. Step S4 calculates the ischemic time interval of the ischemic event based on the stored start time Tᵢ^{st} and an end time Tᵢ^{e}, Tᵢ=Tᵢ^{e}−Tᵢ^{st}. Alternatively, the ischemic time interval is obtained by reading the now stopped ischemia trigger. Step S4 optionally also initiates a recovery timer.

A next step S5 investigates whether the following recovery period has ended. A detected recovery end triggers a next step S6 to determine a recovery time interval based on the determined end time Tᵣ^{e} of the recovery period and the end time of the ischemic event, Tᵣ=Tᵣ^{e}−Tᵢ^{e}. Alternatively, the now stopped recovery timer is read to get the recovery time intervals.

The two determined time intervals are preferably stored in step S7 and co-processed in step S8 for monitoring the ischemic status of the subject as previously described.

The method then ends or returns to step S1 for detecting a further potential ischemic event of the subject's heart, which is schematically illustrated by the line L1.

FIG. 9 is a flow diagram illustrating an additional, optional step of the ischemia monitoring method. The method continues from step S7 of FIG. 8. A next step S10 calculates a statistical distribution of the currently determined ischemia and recovery time interval pair together with corresponding time interval pairs determined for previously detected ischemic events of the subject's heart. The calculated statistical distribution, typically represented in the form of mean values and standard deviations is compared in step S8 of FIG. 8 with a corresponding reference statistical distribution and its reference mean values and standard deviations as previously described.

FIG. 10 is a flow diagram illustrating another optional step of the ischemia monitoring method applicable in the case multiple so-called ischemia severity curves are available and representing the recovery time interval as a function of ischemic time interval for different ischemia severity levels. The method then continues from step S7 of FIG. 8. A next step S20 identifies one of the multiple ischemia severity curves based on the determined ischemia and recovery time intervals. The ischemia severity classification assigned to the ischemia severity curve identified as being most relevant for the current time interval pair is used in step S8 of FIG. 8 for monitoring the ischemic status of the subject and preferably for detecting any change in the ischemic status.

FIG. 11 is a flow diagram illustrating an additional, optional step of the ischemia monitoring method. The method continues from step S6 of FIG. 8, with the next step S30 determining an activity level of the subject in addition to the determination of the time interval pair. The activity level data is preferably stored together with the time interval data in step S7 of FIG. 8 and used with the time interval data for the ischemic status monitoring as described herein.

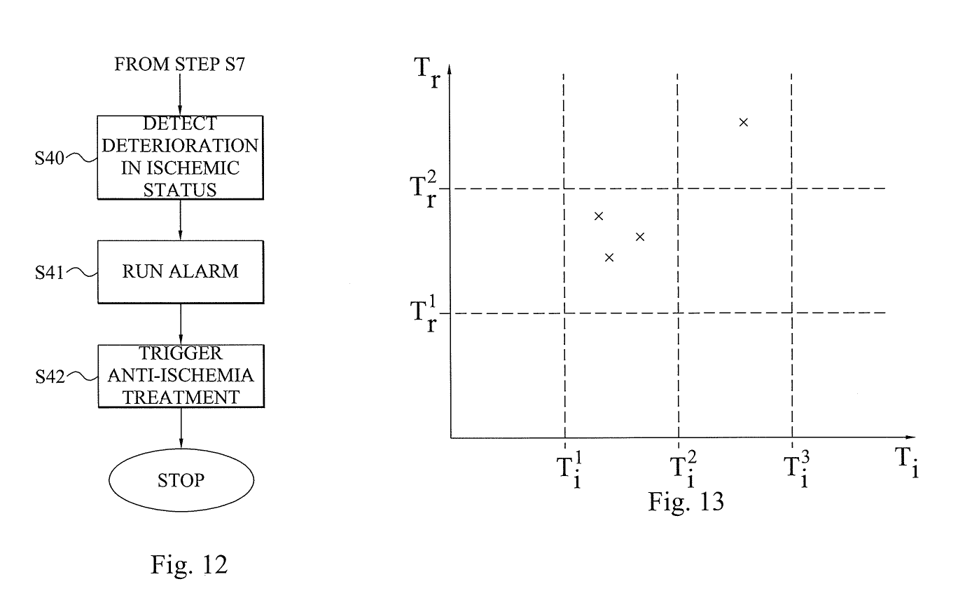

FIG. 12 is a flow diagram illustrating an embodiment of the ischemic status monitoring step S8 of FIG. 8 in more detail. The method continues from step S7 in FIG. 8. A next step S40 detects a deterioration in ischemic status of the subject based on the ischemic and recovery time intervals and optionally the activity level data. In response to this deterioration the following optional step S41 activates an alarm informing the subject or his/her physician of the detected deterioration. In the implanted embodiment, the alarm can be a tactile or audio alarm or trigger an automatic upload of an alarm signal to an external communication unit, such as a home monitor, which in turn can forward the alarm signal to the physician using a computer network, telephone network or a radio-based wireless communication network.

In addition to or instead of running an alarm in step S41, an anti-ischemia treatment is triggered in step S42 in response to the deterioration detection. This treatment trigger is preferably an automatic trigger, which can, for instance, initiate delivery of anti-ischemic electric treatment pulses, an adjustment of the operation characteristics or functions of an IMD and/or the release of anti-thrombotic and/or other anti-ischemic agents from the IMD.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

We claim as our invention:

1. An ischemia monitoring system comprising:
an ischemia onset detector that detects an onset of an ischemic event of a tissue of a subject;
an ischemia end detector that detects an end of said ischemic event;
a recovery end detector that detects an end of a recovery from said ischemic event;
a time processor configured to determine an ischemic time interval of said ischemic event based on said onset of said ischemic event detected by said ischemia onset detector and said end of said ischemic event detected by said ischemia end detector and to determine a recovery time interval of said recovery based on said end of said ischemic event detected by said ischemia end detector and said end of said recovery detected by said recovery end detector;
a memory controller that stores said ischemic time interval and said recovery time interval determined by said time processor or a parameter derivable from said ischemic time interval and said recovery time interval in a connected memory; and
a status processor configured to determine an ischemic status of said subject based on said ischemic time interval and said recovery time interval determined by said time processor.

2. The system according to claim 1, wherein said status processor is configured to detect a change in ischemic status of said subject based on said ischemic time interval and said recovery time interval determined by said time processor.

3. The system according to claim 1, wherein said ischemia onset detector detects onset of N−1 further ischemic events of said tissue (10), said ischemia end detector detecting the respective ends of said N−1 further ischemic events and said recovery end detector detects end of recovery from said N−1 further ischemic events, where N≧2, said system further comprising a distribution calculator configured to calculate a statistical distribution of said N pairs of ischemic time intervals and recovery time intervals, and wherein said status processor is configured to determine said ischemic status of said subject based on a comparison of said statistical distribution calculated by said distribution calculator with a reference statistical distribution.

4. The system according to claim 1, further comprising:
a curve provider that provides multiple ischemia severity curves of recovery time intervals versus ischemic time intervals, said multiple ischemia severity curves having different recovery time intervals for each ischemic time interval; and
a curve identifier configured to identify an ischemia severity curve among said multiple ischemia severity curves provided by said curve provider based on said ischemic time interval and said recovery time interval determined by said time processor, and wherein said status processor is configured to determine said ischemic status of said subject based on an ischemia severity classification assigned to said ischemia severity curve identified by said curve identifier.

5. The system according to claim 1, further comprising an activity sensor that determines an activity level of said subject in connection with at least one of said ischemia onset detector detecting said onset of said ischemic event, said ischemia end detector detecting said end of said ischemic event and said recovery end detector detecting said end of a recovery from said ischemic event, wherein said status processor is arranged for determining said ischemic status of said subject based on said activity level determined by said activity sensor and said ischemic time interval and said recovery time interval determined by said time processor.

6. An implantable medical device comprising:
at least one cardiac lead;
multiple electrodes arranged for collecting electrical signals from a heart of a subject, at least one of said multiple electrodes being arranged on said at least one cardiac lead;
an ischemia managing system connected to at least one electrode among said multiple electrodes, and comprising an ischemia onset detector that detects an onset of an ischemic event of a tissue of a subject, an ischemia end detector that detects an end of said ischemic event, a recovery end detector that detects an end of a recovery from said ischemic event, a time processor configured to determine an ischemic time interval of said ischemic event based on said onset of said ischemic event detected by said ischemia onset detector and said end of said ischemic event detected by said ischemia end detector and to determine a recovery time interval of said recovery based on said end of said ischemic event detected by said ischemia end detector and said end of said recovery detected by said recovery end detector, a memory controller that stores said ischemic time interval and said recovery time interval determined by said time processor or a parameter derivable from said ischemic time interval and said recovery time interval in a connected memory, and a status processor configured to determine an ischemic status of said subject based on said ischemic time interval and said recovery time interval determined by said time processor;
a treatment unit configured to generate an electric treatment signal applicable to at least a portion of said heart via two electrodes among said multiple electrodes; and
a treatment controller configured to control operation of said treatment unit responsive to said status processor detecting a change in said ischemic status of the subject.

7. The device according to claim 6, comprising an intracardiac electrogram, (IECG) processor configured to generate an IECG based on said electric signals collected by said multiple electrodes from said heart, said ischemia onset detector being configured to detect said onset of said ischemic event based on presence of a ST-deviation in said IECG generated by said IECG processor and said ischemia end detector being configured to detect said end of said ischemic event based on ceasing of said ST-deviation in said IECG generated by said IECG processor.

8. The device according to claim 7, wherein
said IECG processor is configured to generate multiple IECGs based on electric signals collected by multiple combinations of at least two electrodes of said multiple electrodes from said heart and to calculate a pseudo-global IECG from said multiple IECGs;
said ischemia onset detector (110) is arranged for detecting said onset of said ischemic event based on presence of a ST-deviation in said pseudo-global IECG generated by said IECG processor (220); and
said ischemia end detector (120) is arranged for detecting said end of said ischemic event based on ceasing of said ST-deviation in said pseudo-global IECG generated by said IECG processor (220).

9. The device according to claim 6, comprising:
a signal generator configured to generate an electric signal that is applied over at least a portion of said heart by two electrodes of said multiple electrodes; and
an impedance processor configured to determine impedance data reflective of mechanical function of at least a portion of said heart based on said electric signal generated by said signal generator and a resulting electric signal collected by two electrodes among said multiple electrodes, said recovery end processor being configured to detect said end of said recovery from said ischemic event based on said impedance data.

10. The device according to claim 9, wherein said impedance processor is configured to determine said impedance data based on said electric signal generated by said signal generator and a resulting electric signal collected by two electrodes among said multiple electrodes during a diastolic phase of a heart cycle of said heart.

11. The device according to claim 6, comprising an alarm unit configured to emit at least one of a tactile alarm and an audio alarm in response to said status processor detecting a deterioration in said ischemic status of said subject.

12. (canceled)

13. The device according to claim 6, wherein said treatment controller is configured to reduce a maximum tracking rate of said treatment unit in response to said status processor detecting a change in said ischemic status of said subject.

14. An ischemia monitoring method comprising:
detecting an onset of an ischemic event of a tissue of a subject;
detecting an end of said ischemic event;
determining an ischemic time interval of said ischemic event based on said onset of said ischemic event and said end of said ischemic event;
detecting an end of a recovery from said ischemic event;
determining a recovery time interval of said recovery based on said end of said ischemic event and said end of said recovery;
storing said ischemic time interval and said recovery time interval or a parameter derivable from said ischemic time interval and said recovery time interval; and
monitoring an ischemic status of said subject based on said ischemic time interval and said recovery time interval.

15. The method according to claim 14, wherein said monitoring step comprises detecting a change in ischemic status of said subject based on said ischemic time interval and said recovery time interval.

16. The method according to claim 14, further comprising repeating said detecting steps and said determining steps for N−1 further ischemic events of said tissue to obtain N pairs of ischemic time intervals and recovery time intervals, where N≧2, wherein said step of detecting said change comprises:
calculating a statistical distribution of said N pairs of ischemic time intervals and recovery time intervals; and
monitoring said ischemic status of said subject based on a comparison of said statistical distribution with a reference statistical distribution.

17. The method according to claim 14, wherein said monitoring step comprises:
providing multiple ischemia severity curves of recovery time intervals versus ischemic time intervals, said multiple ischemia severity curves having different recovery time intervals for each ischemic time interval;
identifying an ischemia severity curve of said multiple ischemia severity curves based on said ischemic time interval and said recovery time interval; and
monitoring said ischemic status of said subject based on an ischemia severity classification assigned to said identified ischemia severity curve.

18. The method according to claim 14, further comprising:
determining an activity level of said subject in connection with at least one of detecting said onset of said ischemic event, detecting said end of said ischemic event and detecting said end of a recovery from said ischemic event; and
monitoring said ischemic status of said subject based on said activity level and said ischemic time interval and said recovery time interval.
